(19) United States
(12) Patent Application Publication
(10) Pub. No.: US 2022/0151839 A1
(43) Pub. Date: May 19, 2022

(54) DIAPER WITH COLLECTION POUCH

(71) Applicant: EASYDAY HEALTH PRODUCTS INC., Blainville (CA)

(72) Inventors: Cristian STEFU, Blainville (CA); Ioana Mihaela HIDISAN, Blainville (CA); Regent RACINE, Blainville (CA)

(21) Appl. No.: 17/439,621

(22) PCT Filed: Mar. 16, 2020
(86) PCT No.: PCT/CA2020/000028

§ 371 Date: Sep. 15, 2021

Related U.S. Application Data
(60) Provisional application No. 62/819,132, filed on Mar. 15, 2019.

(51) Int. Cl. A61F 13/495; A61F 13/551

(52) U.S. Cl. CPC A61F 13/495; A61F 13/5512

(57) ABSTRACT

A diaper comprises a bottom layer and a top layer mounted thereto. The bottom layer defines a top surface and an opposite outer surface and comprises an absorbent core therebetween. The top layer is mounted to the bottom layer about a peripheral border thereof. The top layer defines a top layer area delimited by the peripheral border overtying the top surface and being unconnected to the bottom layer. The top layer area, the top surface and the peripheral border define a pouch therebetween. The top layer area defines a body contacting surface and an opening for providing access to the pouch. When the diaper is worn by the user, the opening is positioned about the anus of a user for providing for fecal matter to enter the porch during defecation.

### CROSS-REFERENCE TO RELATED APPLICATIONS

The present application claims priority on U.S. Provisional Patent Application No. 62/819,132 filed on Mar. 15, 2019 and being incorporated herein by reference in its entirety.

### TECHNICAL FIELD

The present disclosure generally relates to diapers. More particularly but not exclusively the present disclosure relates to a diaper with a collection pouch.

### BACKGROUND

Diapers include both disposable baby diapers and adult diapers. Diapers can be necessary for adults with various conditions, such as incontinence, mobility impairment, severe diarrhea or dementia. A diaper is a type of underwear that allows the wearer to defecate or urinate without the use of a toilet, by absorbing or containing waste products to prevent soiling of outer clothing or the external environment. When diapers become soiled, they require changing, generally by a second person such as a parent or a caregiver. Diapers have a facing layer to be brought into contact with the wearer's skin, an absorbent panel adjacent thereto, and a water-impervious or a water repellant outer layer. Following defecation, prolonged contact of feces with the wearer's skin may have adverse health consequences such as rashes or infections. For caregivers, changing the soiled diapers of adult patients poses many practical challenges as the task is usually extremely messy with fecal matter spilling out of the diapers and onto the sheets of the patient's bed. The ordeal worsens with patients suffering with sever diarrhea and other digestive issues.

### OBJECTS

An object of the present disclosure is to provide a diaper with a collection pouch.

### SUMMARY

In accordance with an aspect of the present disclosure, there is provided a diaper comprising: a bottom layer defining a top surface and an opposite outer surface and comprising an absorbent core therebetween; and a top layer mounted to the bottom layer about a peripheral border thereof defining a top layer area delimited by the peripheral border overlying the top surface and being unconnected to the bottom layer, the top layer area, the top surface and the peripheral border defining a pouch therebetween, the top layer area defining a body contacting surface and an opening for providing access to the pouch, wherein when the diaper is worn by the user, the opening is positioned about the anus of a user for providing for fecal matter to enter the pouch during defecation.

In an embodiment, the opening is surrounded by a border. In an embodiment, the top layer area comprises a lip defining the border and upwardly protruding therefrom and circumscribing the opening. In an embodiment, the lip portion is formed by rolled up material. In an embodiment, the top layer comprises a region of flexible and resilient material for upwardly protruding the border therefrom. In an embodiment, the border is so configured and sized as to be positioned within the intergluteal cleft.

In an embodiment, the top layer is spaced from the top surface of the bottom layer. In an embodiment, the top layer has a top layer surface area and the bottom layer has a bottom layer surface area, the top layer surface area being smaller than the bottom layer surface area. In an embodiment, an underside surface interfacing with the top layer of the bottom surface, the peripheral border defining an inner contour surface at a junction of the underside surface and the top surface of the bottom layer. In an embodiment, at least one of the underside surface, the top surface of the bottom layer and the inner contour surface comprises impermeable material.

In an embodiment, the diaper further comprises a closure for closing the opening following defecation within the pouch. In an embodiment, the closure comprises an adhesive strip of material. In an embodiment, the closure comprises a drawstring fitted within a border surrounding the opening, the border defining border apertures for opposite ends of the drawstring to protrude therefrom for being pulled to close the opening.

In accordance with an aspect of the present disclosure there is provided a diaper kit comprising a plurality of any one of the above diapers.

Other objects, advantages and features of the present disclosure will become more apparent upon reading of the following non-restrictive description of illustrative embodiments thereof, given by way of example only with reference to the accompanying drawings.

### BRIEF DESCRIPTION OF THE DRAWINGS

In the appended drawings:

FIG. 1 is a top plan view of the diaper in accordance with a non-restrictive illustrative embodiment of the present disclosure;

FIG. 2 is a bottom view of the diaper of FIG. 1;

FIG. 3 is lateral sectional view taken about the central axis along the length of the diaper of FIG. 1;

FIG. 4 is a top plan view of a diaper in accordance with another non-restrictive illustrative embodiment of the present disclosure;

FIG. 5 is a top plan view of the diaper of FIG. 4 following usage.

### DETAILED DESCRIPTION OF ILLUSTRATIVE EMBODIMENTS

Generally stated and in accordance with an aspect of the present disclosure, there is provided a diaper comprising a bottom layer and a top layer mounted thereto. The bottom layer defines a top surface and an opposite outer surface and comprises an absorbent core therebetween. The top layer is mounted to the bottom layer about a peripheral border thereof. The top layer defines a top layer area delimited by the peripheral border overlying the top surface and being unconnected to the bottom layer. The top layer area, the top surface and the peripheral border define a pouch therebetween. The top layer area defines a body contacting surface and an opening for providing access to the pouch. When the diaper is worn by the user, the opening is positioned about the anus of a user for providing for fecal matter to enter the pouch during defecation.

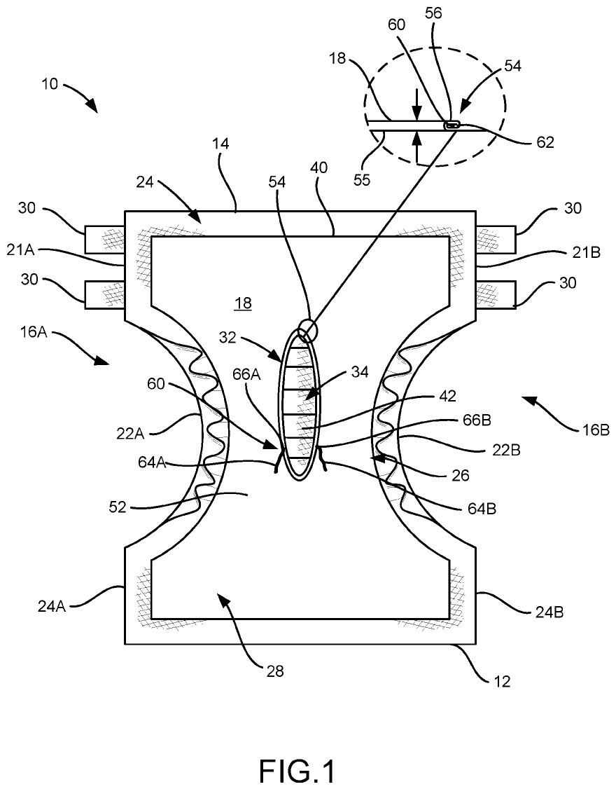

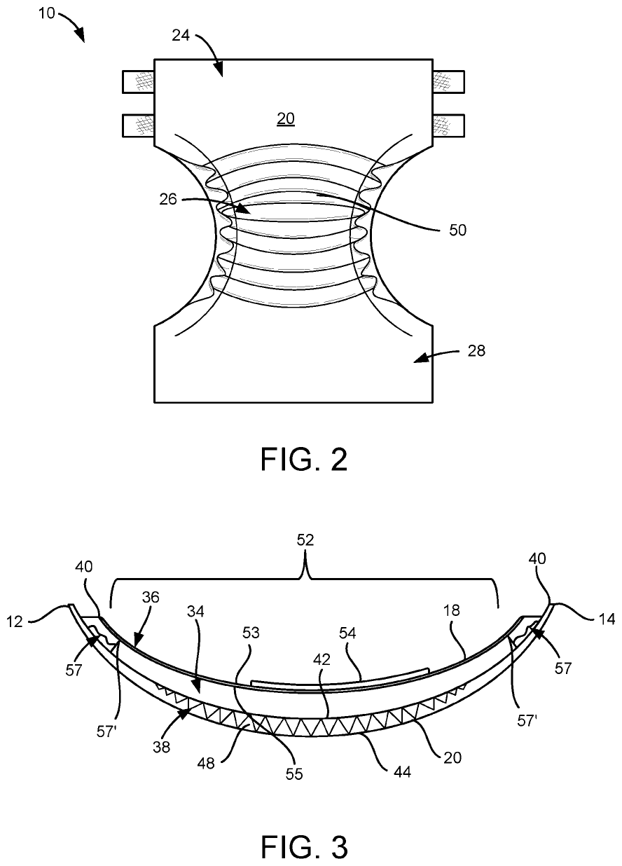

With reference to FIGS. 1 to 5, non-restrictive illustrative embodiment of the present disclosure will now be described.

Referring to FIGS. 1 to 3, the diaper 10 has a generally rectangular shape with front and rear edges 12 and 14, respectively, lateral aides 16A and 168, an inner surface contacting side 18 and an outer side 20.

Each lateral side 16A and 16B has an inwardly curving edges 22A and 22B respectively. Inwardly curved edge 22A is positioned between lateral end edges 21A and 23A, respectively positioned near edges 12 and 14. Similarly, inwardly curved edge 22B is positioned between lateral end edges 21B and 23B, respectively positioned near edges 12 and 14.

The diaper incudes a rear portion 24 (generally formed between later end edges 21A and 21B), a mid portion 26 (generally formed between inwardly curved edges 22A and 22B) and a front portion 28 (generally formed between lateral end edges 23A and 23B). Tabs 30 extend from the lateral edges 21A and 21B for being fastened to the outer side 20 of the front position 28 thereby attaching the diaper around a user's waist and providing for the rear and front portions 24 and 26 to cover the rear and front of the wearer with the mid portion 26 extending therebetween. Of course, belt loops instead of tabs 30 may be used. The inwardly curving edges 22A and 22B are designed to fit around a user's legs and thereby provide a snug fit.

As shown the inner side 18 defines an opening 32 at the mid portion 26 providing access to a pouch 34.

The diaper 10 includes a top layer 36 with a bottom layer 38 mounted thereto about a peripheral border 40 thereof defining the pouch 34 therebetween.

The bottom layer 38 includes a top surface 42, an opposite outer surface 44 with an absorbent core 48 therebetween. The surfaces 42 and 44 and the core 48 are fused together to form a single layer laminate 38. The outer surface 44 includes elastic material 50 causing the diaper to have a c-shaped curved body form between edges 12 and 14.

The top layer 36 defines a top layer area 52 delimited by the peripheral border 40. The top layer 36 is unconnected to the bottom layer 38 and as thus loosely overlies the top surface 42 of the bottom layer 38. The opening 32 is formed in the top layer area 52 providing access to the pouch 34. The top layer 36 includes an undersurface 55.

The undersurface 55 of the top layer 36 and the top surface 42 of the bottom layer 38 are connected at a contour portion 57 near the border 40 defining an inner contour surface 57′. The pouch 34 is thus delimited by surfaces 42, 55 and 57′. The bottom layer top surface 42 defines the bottom border to the pouch 34. The top layer undersurface 55 defines the top border of the pouch 34. The contour portion inner surface 57′ defined the peripheral border of the pouch 34 and is at a junction between the underside surface 55 and the top surface 42 of the bottom layer 38. In an embodiment, surfaces 42, 55 and 57′ are made of impermeable material.

The top layer 36 has a smaller surface area than the bottom layer 38 and as such when mounted thereto at its border 40, the top layer area 52 is stretched causing it to rise upwardly and be spaced above the top surface 42 of the bottom layer 38 bringing the opening 32 upward towards the wearer. The foregoing is further aided by the elastic material 50 which causes the o-shaped configuration of the bottom layer 38. The foregoing configuration and the disposition of the opening 32 provides for positioning the opening 32 about the anus of the wearer for fecal matter to be guided therethrough during defecation.

A lip 54 circumscribes the opening 32. The lip 54 is provided by rolled up material 56 upwardly protruding from the top layer area 52. The lip 54 may be so configured and sized so as to be positioned in the intergluteal cleft. The configuration of the diaper 10 due to the differential in the surface area between the top 36 and bottom 38 layers provides for urging the lip 54 about the anus of the wearer for providing to guide fecal matter through the opening 32 and into the pouch 34 during defecation.

The fecal matter thus fills the pouch 34 formed between the top and bottom layers, 36 and 38, respectively. The lip 54 thus forms a border that substantially blocks spillage of fecal matter onto the top layer surface area 52.

The diaper 10 provided for the focal matter to not be in direct contact with the skin of the wearer post defecation and prior removal of the diaper. Moreover, during removal of diaper 10 following defecation, there is less risk of the caregiver coming into direct contact with the fecal matter as it is all or mostly in the pouch 34. The diaper 10 may be provided with a closure for closing the opening 32 post usage to further avoid outward spillage.

In an embodiment, the closure includes a strip of adhesive material 58 shown in FIG. 5 for covering the opening 32. In an embodiment and with reference to FIG. 1, the closure is a drawstring 60 positioned within the open center 62 of the rolled up material 56. The lip 54 defines the border of the opening 32. The drawstring 60 has two opposite ends 64A and 64B protruding from openings or apertures 66A and 68B in the lip 54 allowing the caregiver to pull on them to close the opening 32.

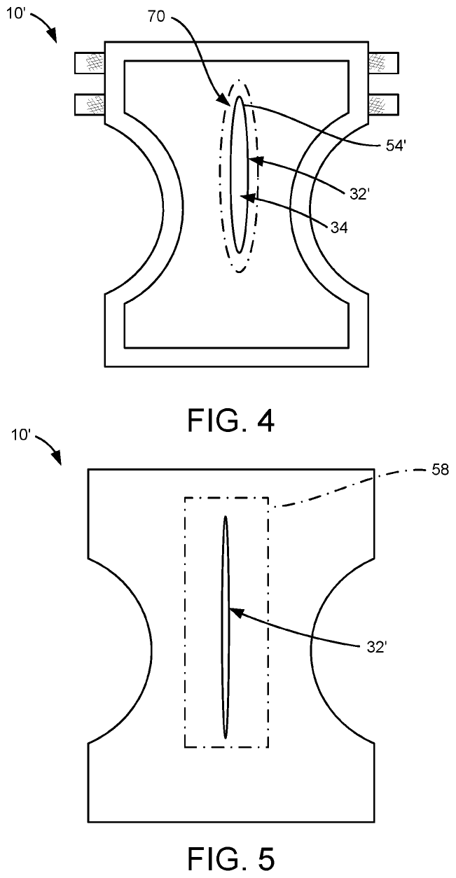

FIGS. 4 and 5 show a diaper 10′ with an opening 32′ having an elongate slit like configuration.

FIG. 4 shows the diaper 10′ prior use and FIG. 5 shows the diaper 10′ post-use containing fecal matter. FIG. 4 shows the border 54′ of the opening 32′ being surrounded by a region flexible and resilient material 70 that upwardly biases the border 54′ towards the anus of the wearer as previously explained. The material 70 can comprises elastomeric material for example covered by a soft surface material so as not to irritate the user. The caregiver can close the opening 32′ with an adhesive strip of material 58 as discussed above.

The diapers 10 and 10′ can be made of a variety of suitable materials and designed in a variety of suitable configurations within the context of the present disclosure.

The various features described herein can be combined in a variety of ways within the context of the present disclosure so as to provide still other embodiments. As such, the embodiments are not mutually exclusive. Moreover, the embodiments discussed herein need not include all of the features and elements illustrated and/or described and thus partial combinations of features can also be contemplated. Furthermore, embodiments with less features than those described can also be contemplated. It is to be understood that the present disclosure is not limited in its application to the details of construction and parts illustrated in the accompanying drawings and described hereinabove. The disclosure is capable of other embodiments and of being practiced in various ways. It is also to be understood that the phraseology or terminology used herein is for the purpose of description and not limitation. Hence, although the present disclosure has been provided hereinabove by way of non-restrictive illustrative embodiments thereof, it can be modified, without departing from the scope, spirit and nature thereof and of the appended claims.

What is claimed is:

1. A diaper comprising:
a bottom layer defining a top surface and an opposite outer surface and comprising an absorbent core therebetween; and
a top layer mounted to the bottom layer about a peripheral border thereof defining a top layer area delimited by the peripheral border overlying the top surface and being unconnected to the bottom layer, the top layer area, the top surface and the peripheral border defining a pouch therebetween, the top layer area defining a body contacting surface and an opening for providing access to the pouch,
wherein when the diaper is worn by the user, the opening is positioned about the anus of a user for providing for fecal matter to enter the pouch during defecation.

2. A diaper according to claim 1, wherein the opening is surrounded by a border.

3. A diaper according to claim 2, wherein the top layer area comprises a lip defining the border and upwardly protruding therefrom and circumscribing the opening.

4. A diaper according to claim 3, wherein the lip portion is formed by rolled up material.

5. A diaper according to any one of claims 2 to 4, wherein the top layer comprises a region of flexible and resilient material for upwardly protruding the border therefrom.

6. A diaper according to any one of claims 2 to 5, wherein the border is so configured and sized as to be positioned within the intergluteal cleft.

7. A diaper according to any one of claims 1 to 6, wherein the top layer is spaced from the top surface of the bottom layer.

8. A diaper according to any one of claims 1 to 7, wherein the top layer has a top layer surface area and the bottom layer has a bottom layer surface area, the top layer surface area being smaller than the bottom layer surface area.

9. A diaper according to any one of claims 1 to 8, wherein the top layer defines an underside surface interfacing with the top layer of the bottom surface, the peripheral border defining an inner contour surface at a junction of the underside surface and the top surface of the bottom layer.

10. A diaper according to claim 9, wherein at least one of the underside surface, the top surface of the bottom layer and the inner contour surface comprises impermeable material.

11. A diaper according to any one of claims 1 to 10, further comprising a closure for closing the opening following defecation within the pouch.

12. A diaper according to claim 11, wherein the closure comprises an adhesive strip of material.

13. A diaper according to claim 11, wherein the closure comprises a drawstring fitted within a border surrounding the opening, the border defining border apertures for opposite ends of the drawstring to protrude therefrom for being pulled to close the opening.

14. A diaper kit comprising:
a plurality of the diaper of any one of claims 1 to 13.
